(19) United States
(12) Patent Application Publication
(10) Pub. No.: US 2023/0247982 A1
(43) Pub. Date: Aug. 10, 2023

(54) Improving Packed Red Blood Cell Storage with a High Viscosity Buffered Storage Solution

(71) Applicant: University of Cincinnati, Cincinnati, OH (US)

(72) Inventors: Bernardin Joseph, Cincinnati, OH (US); Kasiemobi Pulliam, Cincinnati, OH (US); Timothy A Pritts, Cincinnati, OH (US); Salwa Arafa, Liberty Twp, OH (US); Charles Caldwell, Cincinnati, OH (US); Alex Lentsch, Hebron, KY (US); Michael Goodman, Cincinnati, OH (US)

(73) Assignee: University of Cincinnati, Cincinnati, OH (US)

(21) Appl. No.: 18/015,158

(22) PCT Filed: Jul. 13, 2021
(86) PCT No.: PCT/US21/41471

§ 371 Date: Jan. 9, 2023

Related U.S. Application Data
(60) Provisional application No. 63/050,904, filed on Jul. 13, 2020.

(51) Int. Cl. A01N 1/02; C12N 5/078

(52) U.S. Cl. CPC A01N 1/0226; C12N 5/0641

(57) ABSTRACT

A storage solution for packed red blood cells is disclosed where the solution is alkaline and has a viscosity at 4° C. greater than about 20 millipascal seconds. In one embodiment, the storage solution comprises from about 0.1 percent to about 10 percent of hydroxy-propyl-methyl-cellulose. In another embodiment, the storage solution has a pH greater than about 8.

### CROSS REFERENCE TO RELATED APPLICATIONS

This application is a continuation of PCT Application No. PCT/US21/41471 filed Jul. 13, 2021, which claims benefit of U.S. Provisional Application Ser. No. 63/050,904, filed Jul. 13, 2020, which applications are hereby incorporated by reference in their entirety.

### STATEMENT REGARDING FEDERALLY FUNDED RESEARCH OR DEVELOPMENT

This invention was made with government support under GM008478, GM124156, and GM107625, each awarded by the National Institutes of Health. The U.S. Government has certain rights in the invention.

### TECHNICAL FIELD

The present invention relates to methods of storing packed red blood cells.

### BACKGROUND OF THE INVENTION

Hemorrhage remains a leading cause of early and potentially preventable death in patients following traumatic injury. Resuscitation strategies such as early implementation of a massive transfusion protocol and blood product transfusions in a balanced ratio have been shown to have survival benefits. Administration of packed red blood cells (pRBCs), plasma, platelets, and other hemostatic adjuncts are utilized to treat acute post-injury anemia, volume depletion, and coagulopathy. However, the use of packed red blood cells is not without risk. During storage, pRBCs undergo a progressive series of physical and biochemical alterations collectively termed the “red blood cell storage lesion.” These changes include, but are not limited to, decreases in pH, metabolic activity, membrane deformability, and viability, as well as increases in release of extracellular vesicles and free hemoglobin from the stored erythrocytes. The red blood cell storage lesion alters the quality and function of pRBCs and is associated with post-transfusion complications such as acute pulmonary inflammation, thromboembolic events, and increased mortality.

Blood, a non-Newtonian fluid, is thixotropic by nature, with altered viscosity under different shear forces, physiologic, and pathologic states. In states of extreme volume depletion and hemodilution such as hemorrhage, blood viscosity is often too low to maintain sufficient capillary density, with subsequent exacerbation of end organ injury and systemic inflammation. Transfusion of pRBCs is essential to not only restore blood volume and oxygen carrying, but also to increase the viscosity and restore a functional microcirculation via increased capillary perfusion. Unfortunately, transfusing stored pRBC units that have accumulated the red blood cell storage lesion can lead to systemic inflammation and result in microvascular malperfusion. Therefore, a need still exists for a method to store packed red blood cells while reducing the development of the red blood cell storage lesion.

### SUMMARY OF THE INVENTION

The present invention involves a storage solution for packed red blood cells where the solution is alkaline and has a viscosity at 4° C. greater than about 20 millipascal seconds. In one embodiment, the storage solution has a viscosity at 4° C. greater than about 50 millipascal seconds. In another embodiment, the storage solution has a pH greater than about 8. In one embodiment, the storage solution has a pH from about 8.3 to about 8.5.

In one embodiment, the present invention involves a storage solution for packed red blood cells where the storage solution comprises from about 0.1 percent to about 10 percent of hydroxy-propyl-methyl-cellulose. In another embodiment, the storage solution comprises from about 1 percent to about 6 percent of hydroxy-propyl-methyl-cellulose. In one embodiment, the storage solution comprises about 4 percent of hydroxy-propyl-methyl-cellulose. In another embodiment, the storage solution further comprises sodium bicarbonate.

In one embodiment, the present invention involves a storage solution for packed red blood cells where the storage solution consists of sodium citrate, sodium bicarbonate, sodium phosphate, dextrose, adenine, sodium chloride and hydroxy-propyl-methyl-cellulose in concentrations of 15 mM to 25 mM sodium citrate; 10 mM to 15 mM sodium bicarbonate; 7.5 mM to 12.5 mM sodium phosphate; 45 mM to 65 mM dextrose; 1.5 mM to 3.5 mM adenine; 40 mM to 60 mM sodium chloride; and 1 to 6 weight percent hydroxy-propyl-methyl-cellulose. In another embodiment, the storage solution has a pH greater than about 8. In another embodiment, the storage solution has a pH from about 8.3 to about 8.5.

In one embodiment, the present invention involves a storage solution containing 20.0 mM±less than 10% sodium citrate; 12.5 mM±less than 10% sodium bicarbonate; 9.5 mM±less than 10% sodium phosphate; 55.0 mM±less than 10% dextrose; 2.22 mM±less than 10% adenine; 50 mM±less than 10% sodium chloride; and 4.0±less than 10% weight percent hydroxy-propyl-methyl-cellulose.

In another embodiment, the present invention involves a method for storing red blood cells comprising the steps of a) providing a unit of anticoagulated whole blood, b) separating the red blood cells from the whole blood; and c) adding to the separated red blood cells one of the storage solutions described above. In one embodiment, the method involves adding from about 90 ml to 120 ml of said storage solution to said red blood cells. In another embodiment, the method involves adding from about 100 ml to 110 ml of said storage solution to said red blood cells. In one embodiment, the method further involves storing the red blood cells in the storage solution for at least 21 days wherein the red blood cells have a microvesicle accumulation of less than about 400 microvesicles per μL. In another embodiment, the method further involves storing the red blood cells in the storage solution for at least 42 days wherein the red blood cells have a microvesicle accumulation of less than about 250 microvesicles per μL.

### BRIEF DESCRIPTION OF THE DRAWINGS

FIGS. 1A-1B are graphs showing the effect of storage of murine pRBCs stored in AS-3 or EAS-1587 for up to 14 days. FIG. 1A is a graph showing microvesicle counts as determined by flow cytometry. FIG. 1B is a graph showing cell-free hemoglobin accumulation in storage solution.

FIGS. 2A-2C are graphs showing the effect of storage of murine pRBCs in AS-3 or EAS-1587 for 14 days. FIG. 2A is a graph showing Band-3. FIG. 2B is a graph showing phosphatidylserine externalization expression. FIG. 2C is a graph showing hemolysis in response to osmotic stress.

FIGS. 3A-3C are a series of graphs showing the effect of storage of murine pRBCs in AS-3 or EAS-1587 for 14 days. FIG. 3A is a graph showing the percentage of glucose metabolized during storage period. FIG. 3B is a graph showing the erythrocyte (RBC) hemoglobin content. FIG. 3C is a graph showing advanced oxidative protein products (AOPP).

FIGS. 4A-4B are images showing a murine pRBC microscopy smear comparison of units stored in AS-3 (FIG. 4A) and EAS-1587 (FIG. 4B). FIG. 4C is a graph showing forward (FSC) and side (SCC) scatter evaluation of erythrocytes morphology utilizing flow cytometry.

FIGS. 5A-5D are graphs showing inflammatory markers and serum free hemoglobin from mice that underwent hemorrhagic shock and resuscitation with pRBCS stored in AS-3 or EAS-1587. FIG. 5A shows tumor necrosis factor-alpha (TNF-α); FIG. 5B shows macrophage inflammatory protein 1-alpha/CCL3 (MIP-1α). FIG. 5C shows interleukin-6 (IL-6). FIG. 5D shows cell-free hemoglobin.

FIGS. 6A-6C are a series of graphs showing aspects of the red blood cell storage lesion in human pRBCs stored in AS-3 or EAS-1587 for up to 42 days. FIG. 6A is a graph showing microvesicle counts as determined by flow cytometry. FIG. 6B is a graph showing cell-free hemoglobin accumulation in stored units. FIG. 6C is a graph showing percentage of glucose utilized.

FIGS. 7A-7E are a series of graphs showing murine pRBC units stored in AS-3 or with different concentrations of hypromellose (HPM) that were analyzed for aspects of the red blood cell storage lesion. FIG. 7A is a graph showing microvesicle concentrations. FIG. 7B is a graph showing cell free hemoglobin concentration. FIG. 7C is a graph showing advanced oxidation protein product (AOPP) accumulation. FIG. 7D is a graph showing Band 3. FIG. 7E is a graph showing phosphatidylserine surface expression.

FIGS. 8A-8B are a pair of graphs showing a viscosity analysis of (8A) AS-3 and EAS-1857 storage solutions alone and (8B) human pRBCs stored in the storage solutions at storage (4° C.) and physiologic (37° C.) temperatures.

FIGS. 9A-9D are a series of graphs showing the effect of storage of human pRBCs in AS-3 or EAS-1587 for 42 days. FIG. 9A is a graph showing hemolysis in response to osmotic stress. FIG. 9B is a graph showing phosphatidylserine expression. FIG. 9C is a graph showing oxidative stress as determined by advanced oxidation protein products (AOPP). FIG. 9D is a graph showing Erythrocyte (RBC) hemoglobin content.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention.

The present invention involves the development of a novel red blood cell storage solution that leads to a reduction in the red blood cell storage lesion as well as a reduced inflammatory response to resuscitation following hemorrhagic shock. In one embodiment, the solution is alkaline and has a viscosity at 4° C. greater than about 20 millipascal seconds. In another embodiment, the invention comprises a novel solution, termed EAS-1587 (Experimental Additive Solution 1587). We have surprisingly discovered that storage of pRBCs in EAS-1587 resulted in attenuated aspects of the red blood cell storage lesion in human and murine pRBC units as well as a decreased systemic inflammatory response in mice undergoing hemorrhage and resuscitation. Increasing viscosity of the pRBC storage solution is associated with decreased severity of the storage lesion as well as a diminished inflammatory response after resuscitation. The data demonstrated herein shows that the present invention's novel storage solution with increased viscosity and pH will improve the quality of and recipient response to stored pRBCs.

The storage solution and its method of use described herein are useful for the extended storage of packed red blood cells (e.g. approximately 21 days or greater) that have been separated from whole blood. In one embodiment, a red blood cell storage medium is provided that includes nutrients, buffers and salts. The red blood cell storage solution may be an aqueous solution which may include about 15 mM to about 25 mM sodium citrate; about 10 mM to about 15 mM sodium bicarbonate; about 7.5 mM to about 12.5 mM sodium phosphate; about 45 mM to about 65 mM dextrose; about 1.5 mM to about 3.5 mM adenine; about 40 mM to about 60 mM sodium chloride and about 1 to about 6 weight percent hydroxy-propyl-methyl-cellulose. In one embodiment, the storage solution may have a pH greater than about 8.0. In another embodiment, the storage solution may have a pH of from about 8.0 to about 9.0.

As discussed above, in one embodiment of the storage solution, sodium citrate may be present from about 15 mM to about 25 mM. In another embodiment, the sodium citrate may be present from about 17.5 mM to about 22.5 mM. In another embodiment, the sodium citrate may be present at a level of about 20.0 mM±less than 10%.

In one embodiment of the storage solution, sodium bicarbonate may be present from about 10 mM to about 15 mM. In another embodiment, the sodium bicarbonate may be present from about 11 mM to about 14 mM. In another embodiment, the sodium bicarbonate may be present at a level of about 12.5 mM±less than 10%.

In one embodiment of the storage solution, sodium phosphate may be present from about 7.5 mM to about 12.5 mM. In another embodiment, the sodium phosphate may be present from about 8.5 mM to about 11 mM. In another embodiment, the sodium phosphate may be present at a level of about 9.5 mM±less than 10%.

In one embodiment of the storage solution, dextrose may be present from about 45 mM to about 65 mM. In another embodiment, the dextrose may be present from about 50 mM to about 60 mM. In another embodiment, the dextrose may be present at a level of about 55 mM±less than 10%.

In one embodiment of the storage solution, adenine may be present from about 1.5 mM to about 3.5 mM. In another embodiment, the adenine may be present from about 2.0 mM to about 3.0 mM. In another embodiment, the adenine may be present at a level of about 2.22 mM±less than 10%.

In one embodiment of the storage solution, sodium chloride may be present from about 40 mM to about 60 mM. In another embodiment, the sodium chloride may be present from about 45 mM to about 55 mM. In another embodiment, the sodium chloride may be present at a level of about 50 mM±less than 10%.

In one embodiment of the storage solution, hydroxy-propyl-methyl-cellulose may be present from about 0.1 percent to about 10 percent of hydroxy-propyl-methyl-cellulose. In another embodiment, the hydroxy-propyl-methyl-cellulose may be present at a level of about 1 to about 6 weight percent. In another embodiment, the hydroxy-propyl-methyl-cellulose may be present from about 3 to about 5 weight percent. In another embodiment, the hydroxy-propyl-methyl-cellulose may be present at a level of about 4 less than 10% weight percent.

Blood transfusion is the preferred treatment of hemorrhagic shock in order to combat the detrimental effects of ongoing RBC and volume depletion. However, transfusion of pRBCs in not without risks. Pre-transfusion microvesicle and cell free hemoglobin accumulation not only affects the stored pRBCs, but also impacts recipient red blood cell viability, end organ microvasculature, and inflammatory status. Following transfusion, the vulnerable RBCs undergo extravascular and intravascular hemolysis. The toxic components that accumulated during storage and are released following hemolysis in the recipient are cleared via macrophage endocytosis. Unfortunately, when there is a large amount of microvesicle and cell-free hemoglobin content in circulation, the mechanism of removal can be overwhelmed, resulting in reduced clearance of these pro-oxidant and pro-inflammatory components. Previous studies have shown that elevated concentrations of cell-free hemoglobin in the recipients' circulation scavenges endothelial nitric oxide, a key component of maintaining vascular perfusion, and may lead to end-organ damage.

Red blood cells from previously stored whole blood demonstrate attenuated aspects of the red blood cell storage lesion. An important difference between the storage conditions for erythrocytes in whole blood as compared to standard storage conditions is related to the viscosity of the storage medium. The present invention involves the storage of pRBCs in a solution with increased viscosity and a more alkaline pH to produce a reduction in the red blood cell storage lesion and a reduced inflammatory response to resuscitation following hemorrhagic shock. The data presented in the Examples show that this solution, termed EAS-1587 (Experimental Additive Solution 1587), has a significant effect on the onset and severity of the red blood cell storage solution.

In some embodiments, EAS-1587 includes hydroxy-propyl-methyl-cellulose (hypromellose). Hypromellose is a biocompatible cellulose ether that is utilized in the processing and production of food, cosmetics, and pharmaceutical products. It increases the viscosity of solutions by forming a hydrophilic matrix that absorbs and retains water. Hypromellose is commonly utilized in nasal sprays, ophthalmic solutions, and oral tablets and has been safely utilized for injection in endomucosal resection of large colonic polyps as well as experimentally for preparation of injectable nanoparticle hydrogels, and rivaroxaban. Based on its biochemical properties and biocompatibility, hypromellose was determined to be useful as a solution constituent of some embodiments of the present invention in order to increase the viscosity of the modified storage solution.

In one embodiment of the present invention, a novel storage solution with increased viscosity and pH is disclosed. In another embodiment, the storage solution is EAS-1587. As shown in the Examples, storage of pRBCs in EAS-1587 resulted in a reduction in microvesicle and cell-free hemoglobin accumulation in vitro as well as a reduced inflammatory response in vivo. Recipient mice demonstrated a reduction in systemic TNF-α and MIP-1α. Therefore, pRBC storage with EAS-1587 reduces the systemic inflammatory response compared to resuscitation with pRBCs stored in AS-3.

In addition, recent studies have identified an association between transfusion of older pRBCs and increased risk of morbidity and mortality post-transfusion. The results of these studies are, in part, attributed to the toxicity of the erythrocyte storage lesions that accumulate in pRBCs over their storage period. During cold storage, erythrocytes in pRBCs have reduced metabolic activity resulting in a decrease in ATP production. During storage, the mechanisms that protect again oxidative stress are impaired, resulting in an accumulation of reactive oxygen species that contribute to RBC membrane damage. Surprisingly, storage in our novel solution (EAS-1587) results in a significantly increased RBC glucose metabolism, suggesting that metabolic activity was improved in these cells. As shown in the Examples, storage in EAS-1587 resulted in reduced accumulation of advanced oxidative productive products in murine pRBCs, with a trend toward a reduction in human pRBCs.

With increased membrane damage during storage, erythrocytes are unable to maintain their normal biconcave disc shape and demonstrate reduced membrane stability, increased susceptibility to osmotic stress, and increased expression of the senescence signal, phosphatidylserine, on the cell surface. When compared to the standard storage solution, AS-3, pRBCS stored in EAS-1587 demonstrated a greater maintenance of Band 3, reduced phosphatidylserine expression, and reduced osmotic fragility, in mice, while similar in humans. Over the duration of storage, the ability of the red blood cells to maintain homeostasis deteriorates, resulting in acidosis, progressive and persistent microvesicle release, as well as RBC hemolysis. Red blood cells stored in the EAS-1587 solution demonstrated attenuation of these deleterious changes, resulting in reduced microvesicle accumulation and reduced RBC lysis with less free hemoglobin release in mice and humans.

The results detailed below establish that EAS-1587, a novel buffered high viscosity storage solution, leads to reduced accumulation of the red blood cell storage lesion during storage as well as attenuated inflammatory cytokines following resuscitation with aged pRBCs. This data shows that the novel storage solution of the present invention is an improvement over the current standard storage solutions. The present invention attenuates the aging of red blood cells during storage and reduces the sequelae that results from systemic inflammation following blood transfusion.

Any of the disclosed red blood cell storage solutions may be added to concentrated red blood cells prepared from whole blood. In one embodiment, from about 50 mls to about 200 mls of storage solution may be added to concentrated red blood cells prepared from one unit of whole blood. In another embodiment, from about 75 mls to about 180 mls may be added, or from about 90 mls to about 120 mls may be added. In another embodiment, approximately 100 to 110 mls of, and in yet another embodiment approximately 105 ml of the red cell storage solution disclosed herein are added to concentrated red blood cells derived from one unit of whole blood, which may be about 150 to 250 mls of concentrated red blood cells. In another embodiment, the unit of whole blood is about 155 to 185 mls of concentrated red blood cells.

### EXAMPLES

### Methods

Human Blood Banking: Human whole blood was obtained from seven healthy volunteers, after obtaining informed consent, via routine phlebotomy techniques according to a protocol approved by the University of Cincinnati Institutional Review Board. The banked whole blood underwent centrifugation at 1,000 g for 15 minutes. The plasma, buffy coat containing leukocytes were separated and discarded. The erythrocyte pellet was resuspended in storage solution in a 2:9 ratio, and stored for the 42-day Food and Drug Administration (FDA) approved storage duration at 4° C.

Murine Blood Banking: Murine experiments were performed in accordance with a protocol approved by the Institutional Animal Care and Use Committee of the University of Cincinnati. Male 8-10 week old C57BL/6 mice, obtained from the Jackson Laboratory (Bar Harbor, Me.), were acclimated for two weeks in a climate-controlled room with a 12 hour light/dark cycle and fed with standard pellet diet and water ad libitum. Murine blood banking was performed using a modification of our previously characterized protocol. The mice were anesthetized with intraperitoneal pentobarbital (0.1 mg/g body weight) and whole blood was obtained via terminal cardiac puncture. Packed red blood cell units were generated via centrifugation of whole blood at 1,000 g for 15 minutes. The plasma was discarded and the erythrocyte pellet was resuspended in a 2:9 ratio of storage solution and stored for 14 days. Our laboratory has previously investigated and characterized murine blood banking and storage and determined that the 42-day storage duration of human red blood cells is similar to the 14-day storage duration of murine red blood cells.

Erythrocyte Structural and Biochemical Evaluation: Blood smears were utilized to assess the stored erythrocytes for morphological changes that occurred during storage. Findings on the blood smear were confirmed via forward scatter (FSC) and side scatter (SCC) findings on flow cytometry. FSC increases with increased cell size while SCC increases with increased membrane complexity. An i-STAT handheld blood analyzer (Abbott Laboratories, Chicago, Ill.) was utilized to obtain blood gas, electrolyte, and hematologic information. The percentage of glucose metabolized over time was quantified by calculating a percentage change utilizing the glucose concentrations.

Statistical Analysis: GraphPad Prism was utilized (San Diego, Calif.) to perform statistical analysis of data via ANOVA or Student's t-test as noted in the results. P<0.05 was deemed statistically significant. Data is presented as mean f standard error of the mean.

Viscosity measurement: The viscosity of samples was measured via a viscometer (Microvisc, Rheosense Inc., San Ramon, Calif.) at a shear rate of 480 s-1. The samples were assessed at varying temperatures via a temperature control chamber (Microvisc TC, Rheosense Inc., San Ramon, Calif.). 100 μL of AS-3 alone, EAS-1587 alone, pRBCs stored in AS-3, and pRBCs stored in EAS-1587 were analyzed to determine their viscosity at 4° C. and 37° C. in order to determine the effect at (a) storage and (b) body temperature.

### Example 1

Several aspects of the red blood cell storage lesion were evaluated. Red blood cell derived microvesicles were isolated by centrifugation procedures as previously described. Briefly, pRBCs underwent centrifugation at 2,000 g for 10 minutes with collection of the supernatant, further centrifugation at 10,000 g for 10 minutes, with collection of the supernatant and final centrifugation at 21,100 g for 35 minutes to pellet the microvesicles. Microvesicle accumulation was quantified via flow cytometric analysis (Invitrogen Attune NxT flow cytometer (ThermoFisher Scientific, Waltham, Mass.) in mice via phycoerythrin (PE) conjugated rat, anti-mouse Ter-119 antibody (BD Biosciences San Jose, Calif.) binding and in humans via PE conjugated mouse anti-human CD235a (Glycophorin-A) antibody.

Band-3 erythrocyte membrane protein (Band-3) expression and phosphatidylserine externalization expression were assessed via eosin-5′-maleimide (EMA) fluorescence (ThermoFisher Scientific, Waltham, Mass.), and Fluorescein isothiocyanate (FITC) Annexin-V (BD Biosciences San Jose, Calif.) antibody binding respectively. The percentage of red blood cells expressing Band-3 and phosphatidylserine on the membrane surface were quantified via flow cytometry. To determine cell-free hemoglobin content, a marker of erythrocyte lysis, pRBC samples were centrifuged at 21,100 g for 35 minutes, then supernatant cell-free hemoglobin was quantified via a hemoglobin colorimetric assay (BioVision Inc., Milpitas, Calif.) on a microplate spectrophotometer (BioTek Cytation 5, Winooski, Vt.).

In order to evaluate oxidative stress, a commercially available advanced oxidative protein products (AOPP) assay (Cell Biolabs Inc., San Diego, Calif.) was utilized, then analyzed on a microplate spectrophotometer. Susceptibility of red blood cells to osmotic stress was determined by suspending aliquots of erythrocytes in solutions containing increasing concentrations of sodium chloride (0, 0.32, 0.44, 0.56, 0.68, and 0.8% NaCl).

### Example 2

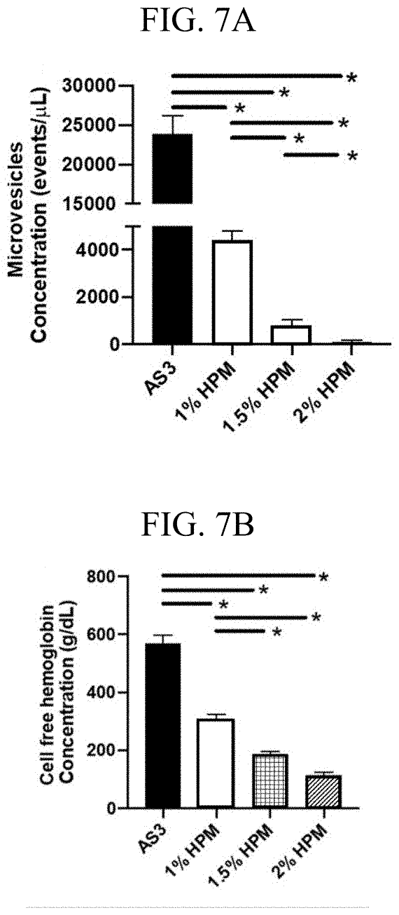

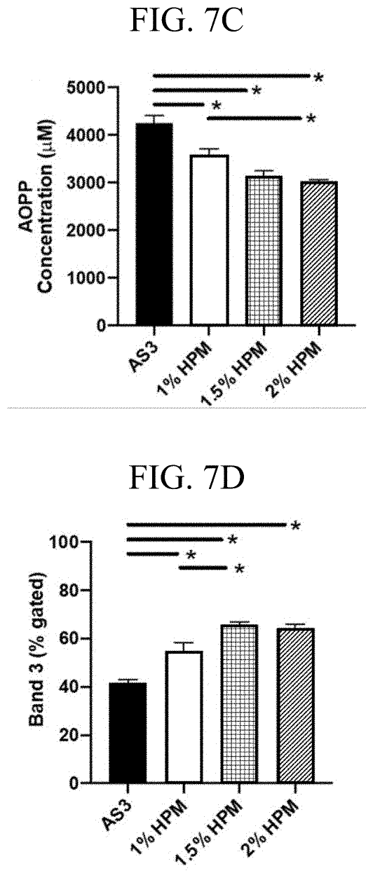

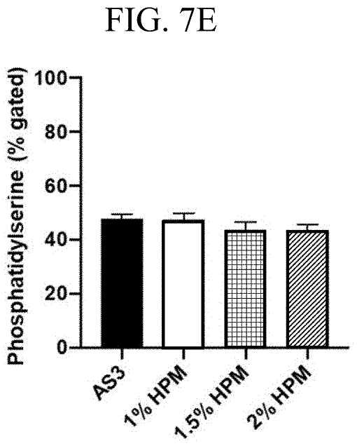

Hypromellose was added to additive solution 3 (AS-3), a standard solution for pRBC storage. Initial experimentation with increasing weight per volume percentages of hypromellose in murine packed red blood cells stored in AS-3 demonstrated that there was a dose dependent reduction in microvesicle release (FIG. 7A), cell-free hemoglobin accumulation (FIG. 7B), and advanced oxidative protein products (FIG. 7C). There was greater retention of Band-3 erythrocyte membrane integrity protein expression in a dose dependent fashion (FIG. 7D). There was no difference in phosphatidylserine externalization (FIG. 7E). Taken together, these data indicate that the addition of hypromellose to AS-3 standard storage solution significantly ameliorated aspects of the red blood cell storage lesion. Use of hypromellose in AS-3 storage solution for human pRBCs produced similar findings, with the exception that the optimal concentration of hypromellose was 4% (data not shown). Therefore, we determined that a 2% and 4% solution of hypromellose were useful for murine and human pRBCs, respectively, and these concentrations were utilized going forward.

During storage, the pH of pRBC units steadily decreases. To address this issue, a modified storage solution was developed with hypromellose to increase the viscosity of the solution and with increased sodium bicarbonate to create a more alkaline pH. Citric acid was not utilized in the modified storage solution in order to minimize acidic components that contribute to a lower pH as the hydrophilic matrices of hypromellose rapidly dissolve when placed in a medium with pH less than or equal to 5.8. The components of AS-3, a standard storage solution, as well as the buffered high-viscosity storage solution, termed experimental additive solution-1587 (EAS-1587) are presented in Table 1.

TABLE 1

| | AS-3 | EAS-1587 |
|---|---|---|
| Components (mM) | | |
| Citric acid | 2 | — |
| Sodium Citrate | 20 | 20.0 |
| Sodium bicarbonate | — | 12.5 |
| Sodium phosphate | 23 | 9.5 |
| Dextrose | 55 | 55 |
| Adenine | 2.22 | 2.22 |
| Sodium Chloride | 70 | 50 |
| Other parameters | | |
| Hypromellose (grams) | — | varies - see text |
| pH | 5.6 | 8.4 |
| Osmolarity (mOsmol/L) | 326 | 291 |

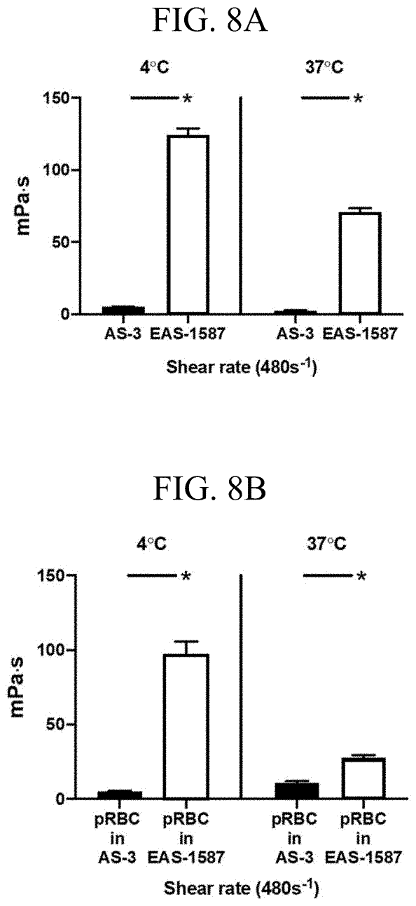

The viscosity of the AS-3 and EAS-1587 storage solutions were analyzed. This analysis showed that the EAS-1587 solution has an increased viscosity compared to AS-3 at both 4° C. and 37° C. (FIG. 8A). Viscosity of human pRBCs stored in EAS-1587, as compared to AS-3, was elevated at 4° C. and 37° C. (FIG. 8B).

### Example 3

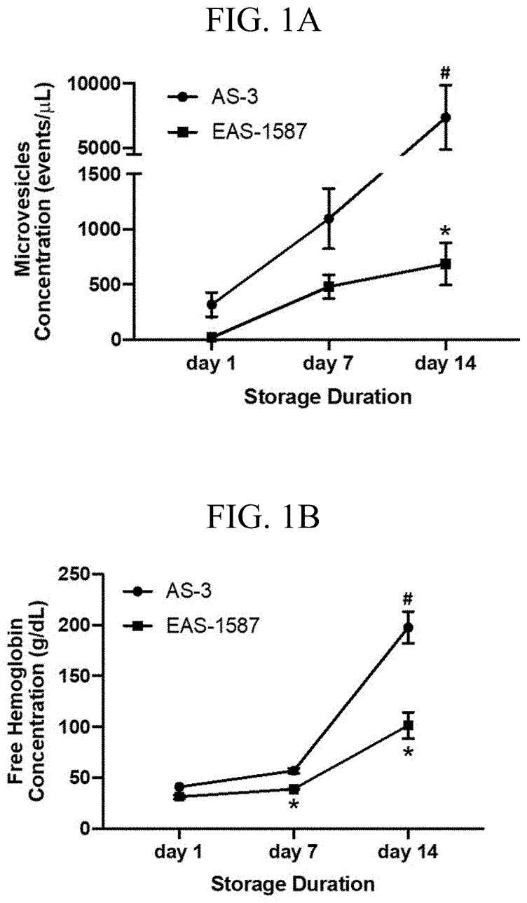

Microvesicle accumulation in pRBC units during storage plays a key role in subsequent lung injury and increased inflammatory response after resuscitation. In order to determine the effect of pRBC storage in EAS-1587 on microvesicle accumulation, murine pRBCs were stored in either AS-3 or EAS-1587 for up to 14 days. Microvesicles were then isolated and quantified. Erythrocyte storage in EAS-1587 was associated with decreased accumulation of pRBC-derived microparticles at days 7 and 14 of storage compared to storage in AS-3. (FIG. 1A).

### Example 4

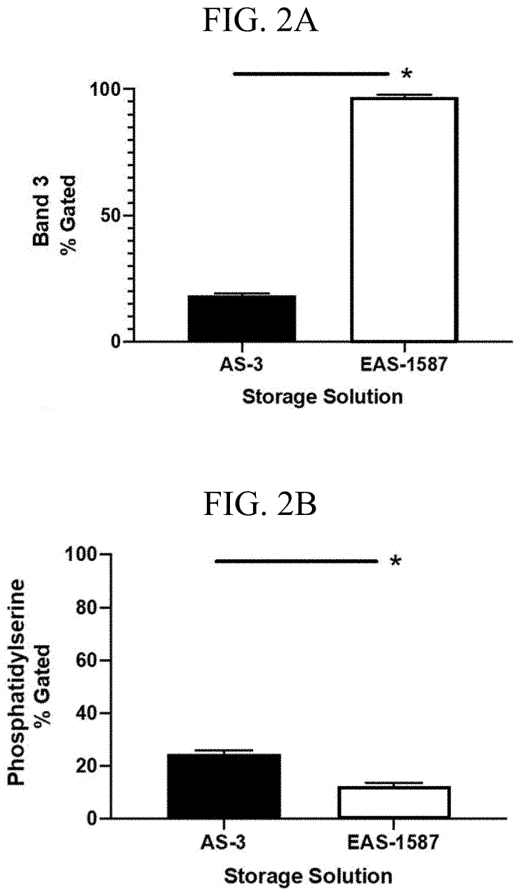

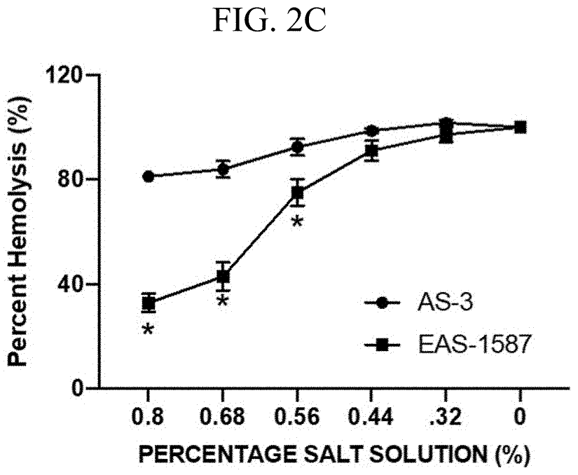

Another potentially harmful aspect of pRBC storage is the accumulation of free hemoglobin, which has been associated with end organ damage after transfusion. When supernatants of pRBCs stored in AS-3 were analyzed, increased free hemoglobin was found during the duration of storage (FIG. 1B). This increase was blunted by storage in EAS-1587 (FIG. 1B). Additional parameters of the red blood cell storage lesion were investigated. It was found that storage in EAS-1587 was associated with greater expression of the membrane protein Band-3 (FIG. 2A), reduced phosphatidylserine externalization (FIG. 2B), and reduced susceptibility to osmotic stress (FIG. 1C) as compared to murine pRBCs stored in AS-3.

### Example 5

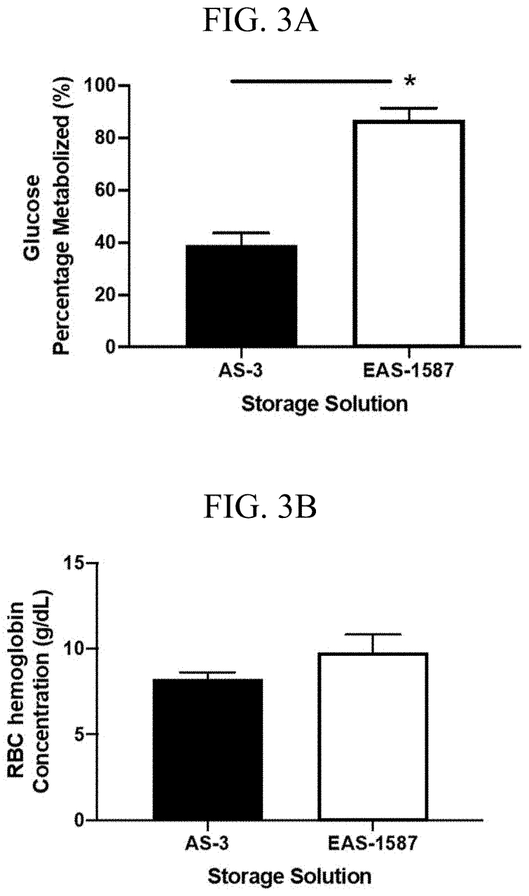

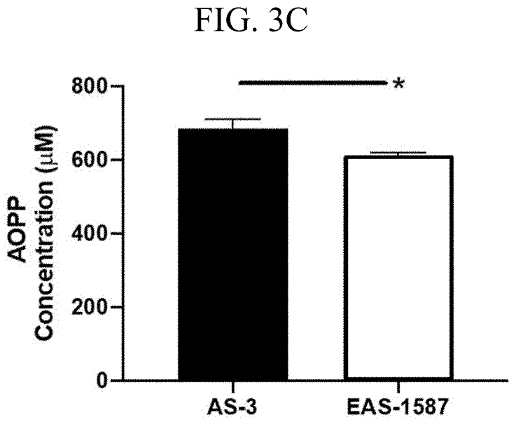

In order to assess the effect of the novel storage solution on glucose metabolism, the percentage of glucose metabolized after 14 days of storage was determined. There was a higher percentage of glucose metabolized in pRBCs stored in EAS-1587 as compared to pRBCs stored in AS-3 (FIG. 3A). There were no differences in intracellular hemoglobin content (FIG. 3B) after EAS-1587 storage. There were also reduced accumulation of advanced oxidative protein products for the pRBCs stored in EAS-1587 (FIG. 3C).

### Example 6

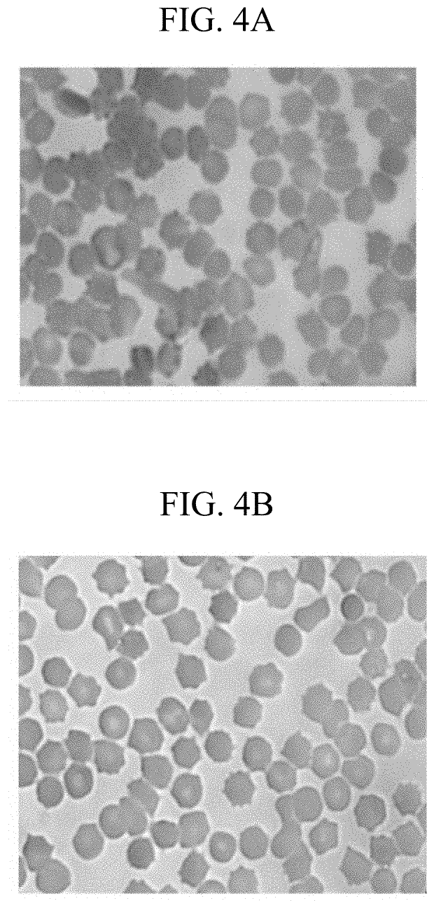

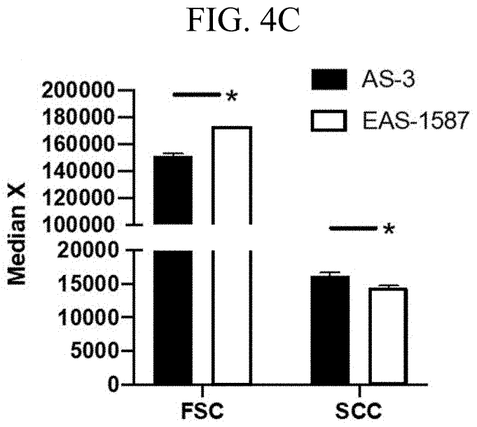

As pRBCs age, the membrane of the erythrocytes undergo structural changes, including loss of discocytic shape and a discocytic-to-spherocytic transformation. Upon examination via peripheral smear, the murine RBCs stored in EAS-1587 had less spheroechinocytic membrane transformation after 14 days of storage (FIGS. 4A and 4B). The peripheral smear findings were confirmed with increased forward scatter and reduced side scatter complexity as determined by flow cytometry for pRBCs stored in EAS-1587 (FIG. 4C). Evaluation of pH, sodium, potassium, ionized calcium, hemoglobin, and hematocrit demonstrated no differences in these parameters (data not shown).

### Example 7

Hemorrhage and resuscitation were carried out. Briefly, 8-10 week old male C57BL/6 mice were anesthetized with intraperitoneal pentobarbital (0.1 mg/gram body weight) followed by groin clipping and sterile preparation with povidone-iodine solution and alcohol. The femoral artery was cannulated with a tapered polyethylene catheter. The catheter was connected to pressure transducers for continuous hemodynamic monitoring of the mice (AD Instruments Lab Chart). Hemorrhagic shock was initiated by withdrawing blood to achieve a mean arterial pressure (MAP) of 25 f 5 mmHg and was maintained for 60 minutes. Following hemorrhagic shock, mice were resuscitated with pRBCs to achieve a MAP greater than 70 mm Hg±5 mm Hg. The mice were monitored for 15 minutes following resuscitation, femoral artery decannulated, and euthanized at 1-hour post procedure end. Sham animals underwent femoral artery cannulation and hemodynamic monitoring for 90 minutes, without hemorrhage or resuscitation.

One hour after hemorrhage and resuscitation, mice were euthanized, and blood obtained via cardiac puncture in a serum separator tube (SST). After 30 minutes, samples were centrifuged at 8,000 rpm for 10 minutes in order to isolate the serum. Serum samples were analyzed for inflammatory chemokines and cytokines as described in the results utilizing a flow cytometry-based cytometric bead array assay (BD Biosciences, San Jose, Calif.).

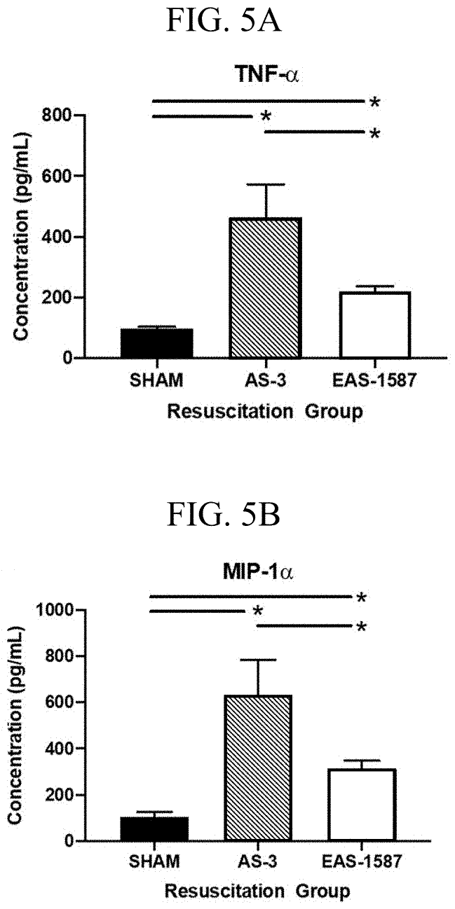

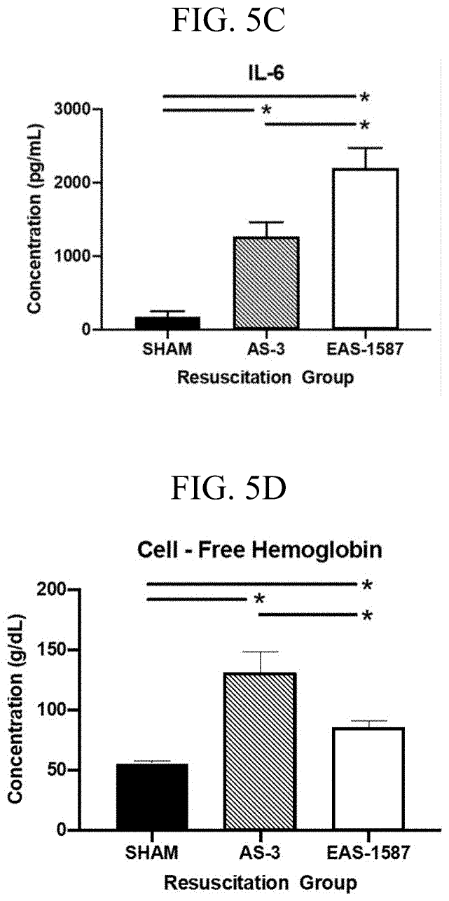

Resuscitation with aged stored pRBCs after hemorrhage is associated with an increased inflammatory response. To determine the effect of resuscitation with pRBCs stored in EAS-1587, mice underwent hemorrhage followed by resuscitation with pRBCs stored in either AS-3 or EAS-1587 for 14 days. The recipient serum demonstrated a reduction in the pro-inflammatory cytokines TNF-α and MIP-1α (FIGS. 5A and 5B). There was an increase of the pleiotropic cytokine IL-6 when compared to resuscitation with AS-3 stored pRBCs (FIG. 5C). The amount of serum-free hemoglobin was lower in mice resuscitated with pRBCs stored in EAS-1587 as compared to those resuscitated with pRBCs stored in AS-3 (FIG. 5D). Taken together, these data indicate that resuscitation with pRBCs stored in EAS-1587 resulted in a decreased inflammatory response.

### Example 8

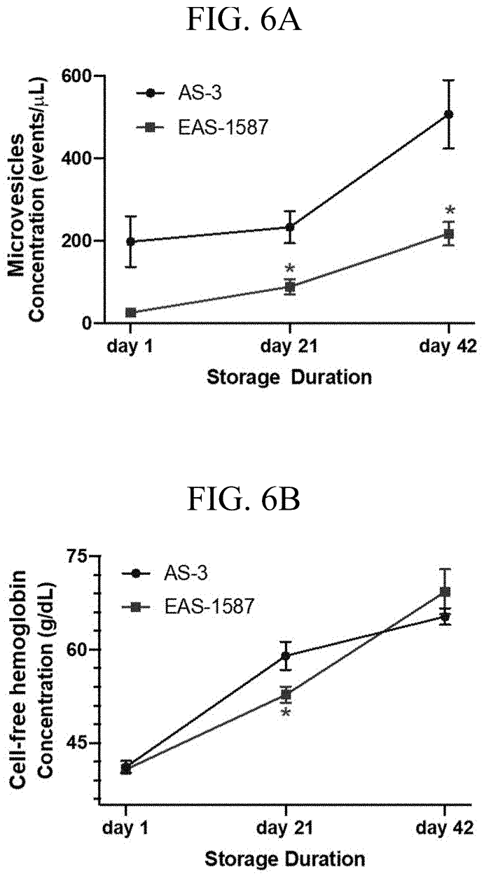

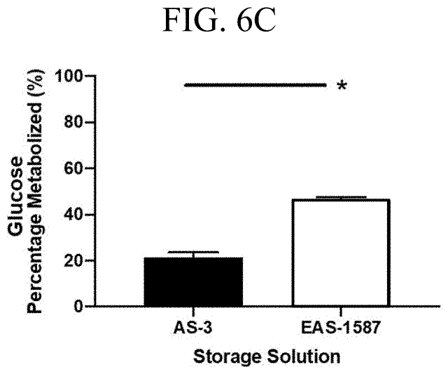

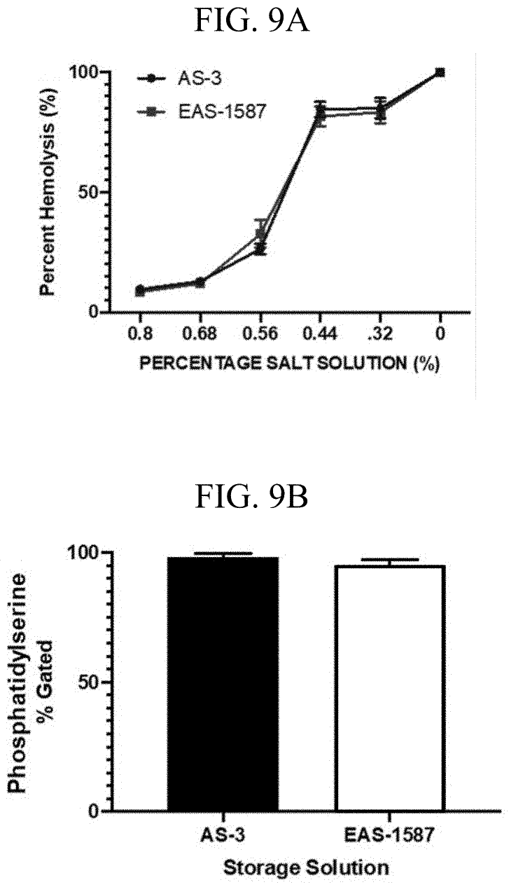

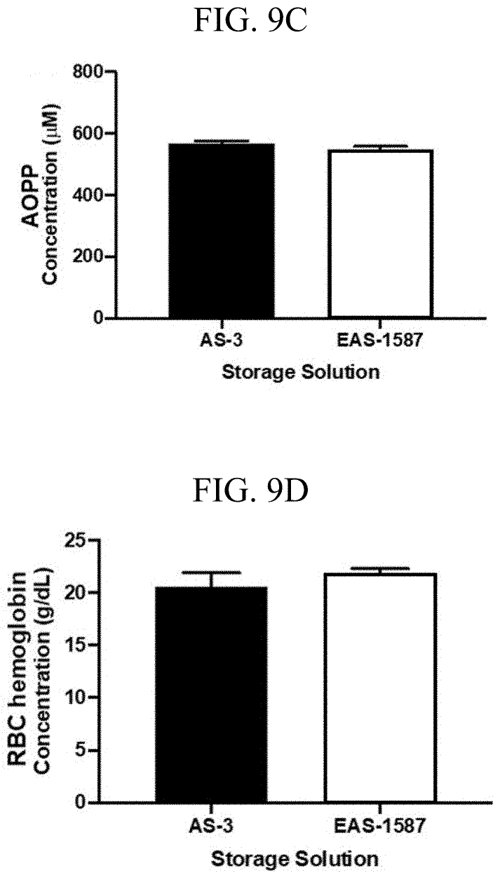

In order to determine the effect of pRBC storage in EAS-1587 on human pRBCs on the red blood cell storage lesion, human pRBCs were stored in either AS-3 or EAS-1587 for up to 42 days, the FDA limit for storage duration. Microvesicles were then isolated and quantified. Erythrocyte storage in EAS-1587 was associated with decreased accumulation of pRBC-derived microparticles at days 21 and 42 of storage compared to storage in AS-3 (FIG. 6A). When supernatants of pRBCs stored in AS-3 were analyzed, increased free hemoglobin was found at day 21 and 42 of storage (FIG. 6B). This increase was blunted at 21 days by storage in EAS-1587 (FIG. 6B). There was no difference at 42 days of storage (FIG. 6B). There was a higher percentage of glucose metabolized in pRBCs stored in EAS-1587 as compared to pRBCs stored in AS-3 (FIG. 6C). There were no significant differences in susceptibility to osmotic stress, expression of Band-3 or phosphatidylserine, accumulation of advanced oxidation protein products, or intracellular erythrocyte hemoglobin content (FIGS. 9A and 9B) between pRBC storage in AS-3 compared to EAS-1587, indicating that the storage solutions were similar in respect to these aspects of storage lesion formation. Evaluation of pH, sodium, potassium, ionized calcium, hemoglobin, and hematocrit demonstrated no differences in these parameters (data not shown). Taken together, these data demonstrate that storage in EAS-1587 mitigates several aspects of the red blood cell storage lesion in human pRBCs.

### Example 9

Blood was obtained from 8-10 week old, C57BL/6 male donor mice, processed, and stored as pRBC units for 14 days in either standard AS-3 storage solution or HV-AS-3 a novel pRBC storage solution with increased viscosity. At the end of the storage duration, pRBCs were analyzed for microvesicle and cell-free hemoglobin content, phosphatidylserine and band-3 erythrocyte membrane integrity protein expression (band-3), and erythrocyte osmotic fragility (EC50). Subsequently, C57BL6 male mice underwent hemorrhagic shock followed by resuscitation with pRBCs stored in either AS-3 or HV-AS-3. After sacrifice, murine serum was analyzed for markers of inflammation.

As compared to AS-3, pRBCs stored in HV-AS-3 demonstrated a significant reduction in microvesicle (7366.9+/−2486.6 vs. 687.8+/−192.9 events/microliter) and cell-free hemoglobin accumulation (197.6+/−15.8 vs. 101.5+/−12.8 g/dL). There was improved band-3 expression (18.6+/−0.61 vs. 96.9+/−0.99% gated) and a reduction in phosphatidylserine expression (24.7+/−1.1 vs. 12.0+/−1.6% gated). The erythrocytes stored in the HV-AS-3 solution demonstrated significantly reduced susceptibility to osmotic stress (EC50 92.3+/−3.1 vs. 75.0+/−5.1%). The serum of mice resuscitated with HV-AS-3-stored pRBCs demonstrated a reduction in macrophage inflammatory protein-alpha (MIP-1a; 630.6+/−153.3 vs. 310.7+/−37.2 pg/ml). This data shows that storage of pRBCs in the storage solution of the present invention mitigated many aspects of the red blood cell storage lesion as well as the inflammatory response to resuscitation following hemorrhage. This modification of the storage solution may foster improvement of pRBC storage and minimize harm incurred upon transfusion.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A storage solution for packed red blood cells wherein the solution is alkaline and has a viscosity at 4° C. greater than about 20 millipascal seconds.

2. The storage solution of claim 1 wherein has a viscosity at 4° C. greater than about 50 millipascal seconds.

3. The storage solution of claim 1 wherein the storage solution has a pH greater than about 8.

4. The storage solution of claim 1 wherein the storage solution has a pH from about 8.3 to about 8.5.

5. A storage solution for packed red blood cells wherein said storage solution comprises from about 0.1 percent to about 10 percent of hydroxy-propyl-methyl-cellulose.

6. The storage solution of claim 5 wherein said storage solution comprises from about 1 percent to about 6 percent of hydroxy-propyl-methyl-cellulose.

7. The storage solution of claim 5 wherein said storage solution comprises about 4 percent of hydroxy-propyl-methyl-cellulose.

8. The storage solution of claim 5 wherein said storage solution further comprises sodium bicarbonate.

9. A storage solution for packed red blood cells wherein said storage solution consists of sodium citrate, sodium bicarbonate, sodium phosphate, dextrose, adenine, sodium chloride and hydroxy-propyl-methyl-cellulose in concentrations of:
a. 15 mM to 25 mM sodium citrate;
b. 10 mM to 15 mM sodium bicarbonate;
c. 7.5 mM to 12.5 mM sodium phosphate;
d. 45 mM to 65 mM dextrose;
e. 1.5 mM to 3.5 mM adenine;
f. 40 mM to 60 mM sodium chloride; and
g. 1 to 6 weight percent hydroxy-propyl-methyl-cellulose.

10. The storage solution of claim 9 wherein the storage solution has a pH greater than about 8.

11. The storage solution of claim 9 wherein the storage solution has a pH from about 8.3 to about 8.5.

12. The storage solution of claim 1 consisting of:
a. 20.0 mM±less than 10% sodium citrate;
b. 12.5 mM±less than 10% sodium bicarbonate;
c. 9.5 mM±less than 10% sodium phosphate;
d. 55.0 mM±less than 10% dextrose;
e. 2.22 mM±less than 10% adenine;
f. 50 mM±less than 10% sodium chloride; and
g. 4.0±less than 10% weight percent hydroxy-propyl-methyl-cellulose.

13. A method for storing red blood cells comprising the steps of:
a. providing a unit of anticoagulated whole blood,
b. separating the red blood cells from the whole blood; and
c. adding to the separated red blood cells the storage solution of claim 1.

14. The method of claim 13 comprising adding from about 90 ml to 120 ml of said storage solution to said red blood cells.

15. The method of claim 13 comprising adding from about 100 ml to 110 ml of said storage solution to said red blood cells.

16. The method of claim 13 further comprising storing said red blood cells in said storage solution for at least 21 days wherein said red blood cells have a microvesicle accumulation of less than about 400 microvesicles per μL.

17. The method of claim 13 further comprising storing said red blood cells in said storage solution for at least 42 days wherein said red blood cells have a microvesicle accumulation of less than about 250 microvesicles per μL.
